(19) United States
(12) Patent Application Publication
(10) Pub. No.: US 2026/0273511 A1
(43) Pub. Date: Sep. 17, 2026

(54) SPHERICAL ALUMINA AND SUPPORTED CATALYST, AND PREPARATION METHODS AND USE THEREOF

(71) Applicants: Tianjin University, Tianjin (CN); Dalian University of Technology, Dalian (CN)

(72) Inventors: Lun Pan, Tianjin (CN); Jijun Zou, Tianjin (CN); Chong Peng, Dalian (CN); Linlin Liu, Tianjin (CN); Kang Xue, Tianjin (CN); Ruijie Gao, Tianjin (CN); Chengxiang Shi, Tianjin (CN); Zhenfeng Huang, Tianjin (CN); Minhua Ai, Tianjin (CN); Xiangwen Zhang, Tianjin (CN)

(73) Assignees: Tianjin University, Tianjin (CN); Dalian University of Technology, Dalian (CN)

(21) Appl. No.: 19/222,272

(22) Filed: May 29, 2025

(30) Foreign Application Priority Data
Mar. 13, 2025 (CN) 202510293825.5

(51) Int. Cl. B01J 21/04; B01J 23/42; B01J 35/30; B01J 35/36; B01J 35/40; B01J 35/51; B01J 35/61; B01J 35/64; B01J 37/00; B01J 37/02; B01J 37/08; B01J 37/18; C01F 7/021; C07C 5/32

(52) U.S. Cl. CPC B01J 21/04; B01J 23/42; B01J 35/36; B01J 35/394; B01J 35/40; B01J 35/51; B01J 35/615; B01J 35/647; B01J 37/0018; B01J 37/0201; B01J 37/088; B01J 37/18; C01F 7/021; C07C 5/325; C07C 2523/56

(57) ABSTRACT

Provided are a spherical alumina and a supported catalyst, and preparation methods and use thereof. A method for preparing the spherical alumina includes: kneading an alumina powder, an adhesive, and water, and subjecting a resulting mixture to extrusion to obtain a strip-shaped sample; and subjecting the strip-shaped sample to spheronization to obtain spherical particles, and subjecting the spherical particles to a first calcination to obtain the spherical alumina. The spherical alumina with a higher sphericity is prepared from the alumina powder using a process that integrates kneading, extrusion, and spheronization, and a diameter of the spherical alumina could be adjusted and controlled by adjusting a diameter of the strip-shaped sample obtained after the extrusion.

### CROSS REFERENCE TO RELATED APPLICATION

This patent application claims the benefit and priority of Chinese Patent Application No. 202510293825.5 filed with the China National Intellectual Property Administration on Mar. 13, 2025, the disclosure of which is incorporated by reference herein in its entirety as part of the present application.

### TECHNICAL FIELD

The present disclosure belongs to the technical field of catalysts, and in particular relates to a spherical alumina and a supported catalyst, and preparation methods and use thereof.

### BACKGROUND

Alumina, as an excellent support material for hydrogenation catalysts, is widely used in the petroleum refining industry. A particle size of a spherical alumina prepared by existing methods is predominantly at a micrometer scale. For example, a micron-sized spherical alumina could be prepared by methods such as primary demagnetization and flame spraying. However, the micron-sized spherical alumina prepared by the existing methods is primarily used as an inorganic filler in the production of ceramic materials and glass fibers. It is difficult to apply in fixed-bed reactors due to issues such as excessive bed pressure drop and bed clogging.

### SUMMARY

In view of this, the present disclosure provides a spherical alumina and a supported catalyst, and preparation methods and use thereof. The spherical alumina prepared by the preparation method according to the present disclosure has good sphericity and a controllable diameter that enables the spherical alumina to be directly used in fixed-bed reactors, and the supported catalyst prepared using the spherical alumina provided by the present disclosure as a support has good catalytic activity for both a hydrogenation reaction and a dehydrogenation reaction.

In order to solve the technical problems mentioned above, the present disclosure provides a method for preparing a spherical alumina, including the following steps:
kneading an alumina powder, an adhesive, and water, and subjecting a resulting mixture to extrusion to obtain a strip-shaped sample; and
subjecting the strip-shaped sample to spheronization to obtain spherical particles, and subjecting the spherical particles to a first calcination to obtain the spherical alumina.

In some embodiments, the adhesive includes one selected from the group consisting of an inorganic acid, formic acid, acetic acid, and citric acid;
a mass ratio of the alumina powder to the adhesive is in a range of 100:0.5-6; and
a mass ratio of the alumina powder to the water is in a range of 1:0.5-2.0.

In some embodiments, the extrusion is performed by using a single-screw extruder with auto-feed compression; and
the strip-shaped sample has an average diameter of not less 0.6 millimetres (mm).

In some embodiments, the spheronization is performed by placing the strip-shaped sample into a spheronizer and subjecting the strip-shaped sample to spheronization to obtain the spherical particles.

In some embodiments, the first calcination is conducted at a temperature of 500° C. to 600° C. for 3 hours to 6 hours.

The present disclosure further provides a spherical alumina prepared by the method described in the aforementioned technical solution, where the spherical alumina has an average diameter of not less 0.6 mm, a sphericity of 0.911 to 0.986, and a strength of 6.7 N/particle to 10.7 N/particle.

The present disclosure further provides use of the spherical alumina described in the aforementioned technical solution as a catalyst support.

The present disclosure further provides a supported catalyst, including a support and a catalytically active component loaded on the support, where the support is the spherical alumina described in the aforementioned technical solution, and the catalytically active component includes a noble metal; and
a loading amount of the catalytically active component in the supported catalyst is 0.5 wt % to 4 wt %.

The present disclosure further provides a method for preparing the supported catalyst described in the aforementioned technical solution, including the following steps:
impregnating the spherical alumina as the support with a noble metal salt solution by an incipient wetness impregnation process to obtain a spherical alumina loaded with a noble metal salt; and
subjecting the spherical alumina loaded with the noble metal salt to a second calcination to obtain the supported catalyst.

The present disclosure further provides use of the supported catalyst described in the aforementioned technical solution or the supported catalyst prepared by the method described in the aforementioned technical solution in a hydrogenation reaction or a dehydrogenation reaction in a fixed-bed reactor.

The present disclosure provides a method for preparing a spherical alumina, including the following steps: kneading an alumina powder, an adhesive, and water, and subjecting a resulting mixture to extrusion to obtain a strip-shaped sample; and subjecting the strip-shaped sample to spheronization to obtain spherical particles, and subjecting the spherical particles to a first calcination to obtain the spherical alumina. In the present disclosure, the spherical alumina with a higher sphericity is prepared from the alumina powder by a process that integrates kneading, extrusion, and spheronization, and a diameter of the spherical alumina could be adjusted and controlled by adjusting a diameter of the strip-shaped sample obtained after the extrusion. Furthermore, a supported catalyst could be prepared by using the spherical alumina provided by the present disclosure as a support to load a noble metal. The supported catalyst could be used in a fixed-bed reactor and has good catalytic activity and catalytic efficiency for both hydrogenation reaction and dehydrogenation reaction.

### BRIEF DESCRIPTION OF THE DRAWINGS

FIGS. 1A-1G show actual images of the spherical alumina prepared in Examples 1-7, where FIG. 1A shows the actual image of the spherical alumina prepared in Example 1, FIG. 1B shows the actual image of the spherical alumina prepared in Example 2, FIG. 1C shows the actual image of the spherical alumina prepared in Example 3, FIG. 1D shows the actual image of the spherical alumina prepared in Example 4, FIG. 1E shows the actual image of the spherical alumina prepared in Example 5, FIG. 1F shows the actual image of the spherical alumina prepared in Example 6, and FIG. 1G shows the actual image of the spherical alumina prepared in Example 7; and

FIG. 2 shows nitrogen adsorption/desorption curves of the supported catalysts prepared in Examples 1-7.

### DETAILED DESCRIPTION OF THE EMBODIMENTS

The present disclosure provides a method for preparing a spherical alumina, including the following steps:
kneading an alumina powder, an adhesive, and water, and subjecting a resulting mixture to extrusion to obtain a strip-shaped sample; and
subjecting the strip-shaped sample to spheronization to obtain spherical particles, and subjecting the spherical particles to a first calcination to obtain the spherical alumina.

In the present disclosure, an alumina powder, an adhesive, and water are kneaded and a resulting mixture is subjected to extrusion to obtain a strip-shaped sample. In a specific embodiment of the present disclosure, the alumina powder may have an average particle size of 10 μm to 100 μm, further 30 μm to 80 μm, and still further 38 μm to 75 μm. In a specific embodiment of the present disclosure, the adhesive may include one selected from the group consisting of an inorganic acid, formic acid, acetic acid, and citric acid; and the inorganic acid may include one selected from the group consisting of nitric acid and hydrochloric acid, and a mass concentration of the inorganic acid may be 1-100%, and further 10-98%. In a specific embodiment of the present disclosure, the water may be one selected from the group consisting of deionized water and high-purity water. In a specific embodiment of the present disclosure, a mass ratio of the alumina powder to the adhesive may be in a range of 100:0.5-6, and specifically 100:2, 100:2.4, 100:3, 100:3.5, or 100:5; and a mass ratio of the alumina powder to the water may be in a range of 1:0.5-2.0, and specifically 1:0.5, 1:0.9, 1:1, 1:1.1, 1:1.5, or 1:1.8.

In a specific embodiment of the present disclosure, the kneading may be conducted in a kneader, and the alumina powder is first added into the kneader, and then the adhesive and the water are added thereto; and the kneading may be conducted for 4 minutes to 6 minutes, and specifically 4 minutes, 5 minutes, or 6 minutes.

In a specific embodiment of the present disclosure, the extrusion is performed by using a single-screw extruder with auto-feed compression to obtain the strip-shaped sample, and an extrusion die used in the single-screw extruder with auto-feed compression is a round-hole die; and the strip-shaped sample may have a diameter of not less 0.6 mm, and specifically may have the diameter of 0.8 mm, 1 mm, or 1.2 mm. In the present disclosure, the diameter of the strip-shaped sample is determined by a diameter of a round hold of the round-hole die. In the present disclosure, a diameter of a spherical alumina is adjusted and controlled by adjusting the diameter of the strip-shaped sample, and the diameter of the strip-shaped sample is 0.2 mm to 0.3 mm smaller than that of the spherical alumina.

In the present disclosure, after obtaining the strip-shaped sample, the strip-shaped sample is subjected to spheronization to obtain spherical particles and the spherical particles are subjected to a first calcination to obtain the spherical alumina. In a specific embodiment of the present disclosure, the spheronization is performed by placing the strip-shaped sample into a spheronizer and subjecting the strip-shaped sample to spheronization to obtain the spherical particles; and the spheronizer may be operated with an air blast pressure of 0.2 MPa to 0.22 MPa, a rotation speed of 1,800 rpm to 1, 900 rpm, and a rolling duration of 3 minutes to 4 minutes.

In a specific embodiment of the present disclosure, before the first calcination, the method for the spherical alumina may further include: airing the product obtained after the spheronization in a room-temperature environment, and a temperature of the room-temperature environment may be 20° C. to 35° C., and further 25° C. to 30° C.; and the airing time is not specifically limited in the present disclosure, as long as the solvent in the product obtained after spheronization can be removed.

In a specific embodiment of the present disclosure, the first calcination may be conducted at a temperature of 500° C. to 600° C., and further 550° C. to 580° C.; the first calcination temperature may be conducted for 3 hours to 6 hours, further 4 hours to 5 hours; and the first calcination is conducted in an air atmosphere.

In the present disclosure, the adhesive added during molding (extrusion) and other impurities could be removed via the first calcination, and after the first calcination, γ-type alumina is obtained.

The method provided by the present disclosure features a simple preparation process and a high spheronization rate.

The present disclosure further provides a spherical alumina prepared by the method described in the aforementioned technical solution. The spherical alumina has an average diameter of not less 0.6 mm, further 0.6 mm to 3 mm, and specifically 1 mm, 1, 2 mm or 1.5 mm; the spherical alumina has a sphericity of 0.911 to 0.986, specifically 0.911, 0.932, 0.939, 0.976, 0.981, 0.983, or 0.986; and the spherical alumina has a strength of 6.7 N/particle to 10.7 N/particle, and specifically 6.7 N/particle, 7.0 N/particle, 8.8 N/particle, 9.0 N/particle, 9.5 N/particle, 10 N/particle, or 10.7 N/particle.

The spherical alumina provided by the present disclosure has not only high strength but also uniform and small particle size and is not easy to bond together, which is conducive to improving catalytic performance of a supported catalyst using the spherical alumina as a support; in addition, the spherical alumina provided by the present disclosure has suitable acidity and has high hydrogenation activity and dehydrogenation activity after loading a noble metal.

The present disclosure further provides use of the spherical alumina described in the aforementioned technical solution as a catalyst support.

The present disclosure further provides a supported catalyst, which includes a support and a catalytically active component loaded on the support, where the support is the spherical alumina described in the aforementioned technical solution, and the catalytically active component includes a noble metal, and the noble metal may be platinum or palladium; and a loading amount of the catalytically active component in the supported catalyst is 0.5 wt % to 4 wt %, and specifically 1 wt %, 2 wt %, 3 wt %, or 4 wt %.

The present disclosure further provides a method for preparing the supported catalyst described in the aforementioned technical solution, including the following steps:
impregnating the spherical alumina as the support with a noble metal salt solution by an incipient wetness impregnation process to obtain a spherical alumina loaded with a noble metal salt; and
subjecting the spherical alumina loaded with a noble metal salt to a second calcination to obtain the supported catalyst

In the present disclosure, the spherical alumina as the support is impregnated with a noble metal salt solution by an incipient wetness impregnation process to obtain a spherical alumina loaded with a noble metal salt. In a specific embodiment of the present disclosure, the noble metal salt in the noble metal salt solution may include chloroplatinic acid hexahydrate, tetraammineplatinum nitrate, or palladium chloride. A mass concentration and amount of the noble metal salt solution are not specifically limited in the present disclosure, as long as the loading amount of the noble metal could be met. In a specific embodiment of the present disclosure, the process of impregnating the spherical alumina as the support with the noble metal salt solution could be achieved by spraying or dripping the noble metal salt solution onto the spherical alumina as the support.

In the present disclosure, after obtaining the spherical alumina loaded with the noble metal salt, the spherical alumina loaded with the noble metal salt is subjected to a second calcination to obtain the supported catalyst. In a specific embodiment of the present disclosure, the second calcination may be conducted at a temperature of 300° C. to 500° C., and specifically 350° C., 400° C., or 450° C.; and the second calcination may be conducted for 2 hours to 6 hours, or 3 hours to 4 hours.

In the present disclosure, the second calcination could enhance interaction between a noble metal active phase and the support.

The supported catalyst provided by the present disclosure has high strength, and is not easily broken after a noble metal is loaded via the incipient wetness impregnation method, and has high catalytic efficiency.

The present disclosure further provides use of the supported catalyst described in the aforementioned technical solution or the supported catalyst prepared by the method described in the aforementioned technical solution in a hydrogenation reaction or a dehydrogenation reaction in a fixed-bed reactor. In the present disclosure, the supported catalyst requires reduction activation before being used in the catalytic hydrogenation or the dehydrogenation reaction, and the reduction activation may include reducing the supported catalyst in a reducing atmosphere to obtain an activated catalyst. In a specific embodiment of the present disclosure, the reducing atmosphere may be hydrogen; a pressure of a reduction system may be 0.8 MPa to 1.2 MPa, and specifically 1 MPa; a reduction temperature may be 330° C. to 370° C., and specifically 350° C.; and a reduction time may be 20 hours to 26 hours, further 22 hours to 24 hours. In the present disclosure, a noble metal oxide in the supported catalyst could be reduced to a noble metal through reduction activation.

The present disclosure has no special requirements for using the supported catalyst in a catalytic hydrogenation reaction or a catalytic dehydrogenation reaction, and conventional methods in the art may be adopted.

To further illustrate the present disclosure, the technical solutions provided by the present disclosure will be described in detail below in conjunction with examples, which should not be construed as limiting the scope of the present disclosure, however.

### Example 1

(1) An alumina powder with an average particle size of 75 μm was added to a kneader, then nitric acid with a mass concentration of 68% and deionized water were added to the kneader, and a resulting mixture was stirred at room temperature (25° C.) for 5 minutes to obtain a mixed sample, with a mass ratio of the alumina powder to the nitric acid being 100:1, and a weight ratio of the deionized water to the alumina powder being 1:1.
(2) The mixed sample was placed into a single-screw extruder with auto-feed compression (with an extrusion die having a diameter of 1, 2 mm round holes), and subjected to extrusion to obtain a strip-shaped sample with a diameter of 1.2 mm.
(3) The strip-shaped sample was placed into a spheronizer, and subjected to spheronization to obtain alumina spheres with an average diameter of 1.5 mm. The alumina spheres were naturally aired at room temperature (25° C.) and then calcined in a muffle furnace at 550° C. for 4 hours to obtain a spherical alumina having an average diameter of 1.5 mm, where the spheronization was conducted under conditions: an air blast pressure of 0.2 MPa, a rotation speed of 1, 800 rpm, and a rolling duration of 3 minutes.
(4) An aqueous chloroplatinic acid solution was sprayed onto the spherical alumina by an incipient wetness impregnation process to obtain a spherical alumina loaded with chloroplatinic acid, where a mass concentration and an amount of the aqueous chloroplatinic acid solution were calculated with a Pt loading amount of 1 wt % as a target.
(5) The spherical alumina loaded with the chloroplatinic acid was calcined at 400° C. for 4 hours to obtain a Pt/1.5 mm alumina supported catalyst, with a Pt loading amount of 1 wt %.

### Example 2

(1) An alumina powder with an average particle size of 50 μm was added to a kneader, then acetic acid with a mass concentration of 99.5% and deionized water were added to the kneader, and a resulting mixture was stirred at room temperature (25° C.) for 5 minutes to obtain a mixed sample, with a mass ratio of the alumina powder to the acetic acid being 100:5, and a weight ratio of the deionized water to the alumina powder being 0.9:1.
(2) The mixed sample was placed into a single-screw extruder with auto-feed compression (with an extrusion die having a diameter of 1.0 mm round holes), and subjected to extrusion to obtain a strip-shaped sample with a diameter of 1.0 mm.
(3) The strip-shaped sample was placed into a spheronizer, and subjected to spheronization to obtain alumina spheres with an average diameter of 1, 2 mm. The alumina spheres were naturally aired at room temperature (25° C.) and then calcined in a muffle furnace at 500° C. for 5 hours to obtain a spherical alumina having an average diameter of 1.2 mm, where the spheronization was conducted under conditions: an air blast pressure of 0.22 MPa, a rotation speed of 1, 900 rpm, and a rolling duration of 4 minutes.
(4) An aqueous chloroplatinic acid solution was sprayed onto the spherical alumina by an incipient wetness impregnation process to obtain a spherical alumina loaded with chloroplatinic acid, where a mass concentration and an amount of the aqueous chloroplatinic acid solution were calculated with a Pt loading amount of 2 wt % as a target.
(5) The spherical alumina loaded with chloroplatinic acid was calcined at 450° C. for 4 hours to obtain a Pt/1.2 mm alumina supported catalyst, with a Pt loading amount of 2 wt %.

### Example 3

(1) An alumina powder with an average particle size of 38 μm was added to a kneader, then acetic acid with a mass concentration of 99.5% and deionized water were added to the kneader, and a resulting mixture was stirred at room temperature (25° C.) for 5 minutes to obtain a mixed sample, with a mass ratio of the alumina powder to the acetic acid being 100:3.5, and a weight ratio of the deionized water to the alumina powder being 1:1.
(2) The mixed sample was placed into a single-screw extruder with auto-feed compression (with an extrusion die having a diameter of 0.8 mm round holes), and subjected to extrusion to obtain a strip-shaped sample with a diameter of 0.8 mm.
(3) The strip-shaped sample was placed into a spheronizer, and subjected to spheronization to obtain alumina spheres with an average diameter of 1.0 mm. The alumina spheres were naturally aired at room temperature (25° C.) and then calcined in a muffle furnace at 600° C. for 3 hours to obtain a spherical alumina having an average diameter of 1.0 mm, where the spheronization was conducted under conditions: an air blast pressure of 0.22 MPa, a rotation speed of 1, 900 rpm, and a rolling duration of 3 minutes.
(4) An aqueous chloroplatinic acid solution was sprayed onto the spherical alumina by an incipient wetness impregnation process to obtain a spherical alumina loaded with chloroplatinic acid, where a mass concentration and an amount of the aqueous chloroplatinic acid solution were calculated with a Pt loading amount of 4 wt % as a target.
(5) The spherical alumina loaded with chloroplatinic acid was calcined at 350° C. for 5 hours to obtain a Pt/1.0 mm alumina supported catalyst, with a Pt loading amount of 4 wt %.

### Example 4

A supported catalyst was prepared according to a method in Example 1, except that in step (1), nitric acid with a mass concentration of 68% was replaced with citric acid with a mass concentration of 99%, and a mass ratio of the alumina powder to the citric acid was 100:3; and a Pt loading amount of a resulting Pt/1.5 mm alumina supported catalyst was 3 wt %, and during an incipient wetness impregnation process, a mass concentration and an amount of an aqueous chloroplatinic acid solution were calculated with a Pt loading amount of 3 wt % as a target.

### Example 5

A supported catalyst was prepared according to a method in Example 2, except that in step (2), a diameter of round holes in an extrusion die was 0.8 mm instead of 1.0 mm, and a Pt loading amount of a resulting Pt/1.0 mm alumina supported catalyst was 2 wt %.

### Example 6

A supported catalyst was prepared according to a method in Example 3, except that in step (1), acetic acid with a mass concentration of 99.5% was replaced with nitric acid with a mass concentration of 68%, and a mass ratio of the alumina powder to the nitric acid was adjusted from 100:3 to 100:2.4; in step (2), a diameter of round holes in an extrusion die was adjusted from 0.8 mm to 1.0 mm; and a Pt loading amount of a resulting Pt/1.2 mm alumina supported catalyst was 4 wt %.

### Example 7

A supported catalyst was prepared according to a method in Example 1, except that in step (1), the deionized water was replaced with high-purity water, and a weight ratio of the high-purity water to the alumina powder was adjusted to 1.1:1.

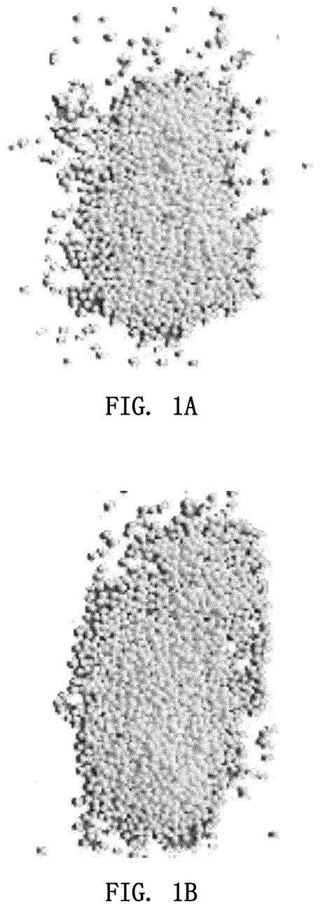

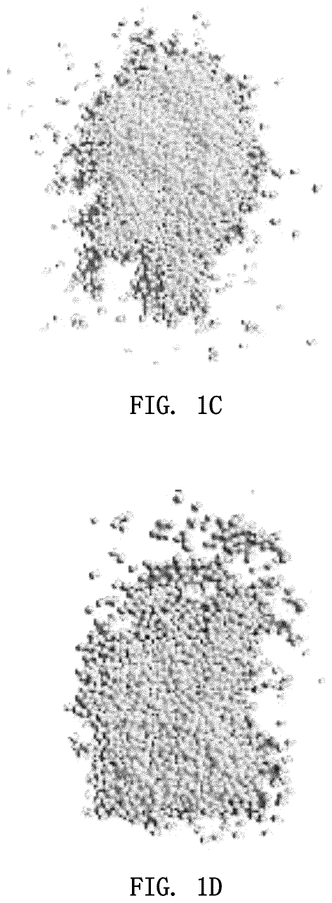

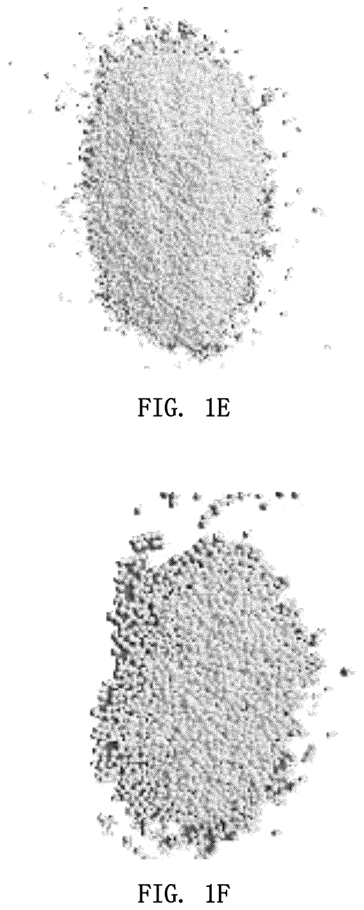

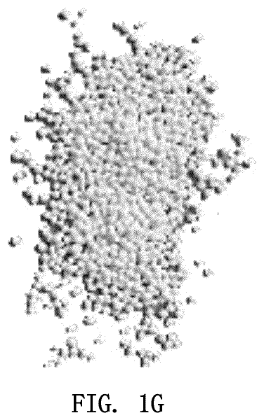

FIGS. 1A-1G show actual images of the spherical alumina prepared in Examples 1-7, where FIG. 1A shows the actual image of the spherical alumina prepared in Example 1, FIG. 1B shows the actual image of the spherical alumina prepared in Example 2, FIG. 1C shows the actual image of the spherical alumina prepared in Example 3, FIG. 1D shows the actual image of the spherical alumina prepared in Example 4, FIG. 1E shows the actual image of the spherical alumina prepared in Example 5, FIG. 1F shows the actual image of the spherical alumina prepared in Example 6, and FIG. 1G shows the actual image of the spherical alumina prepared in Example 7. As can be seen from FIGS. 1A-1G, the spherical alumina prepared by the method of the present disclosure has high sphericity and uniform sizes.

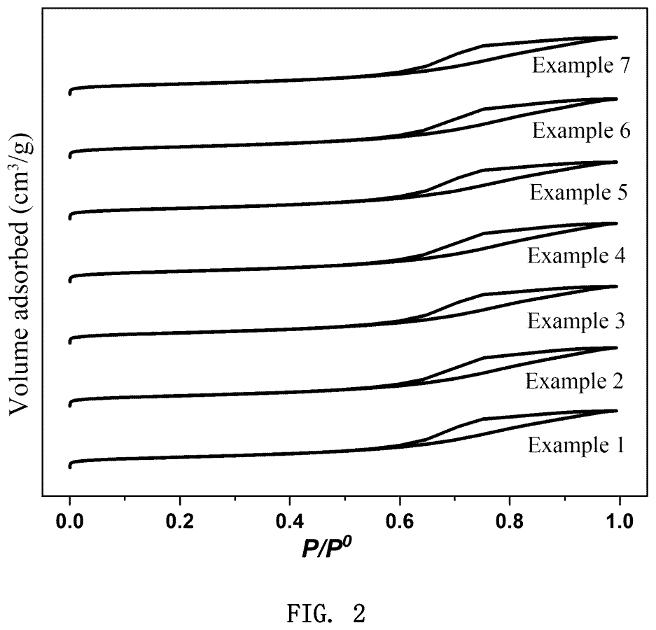

The supported catalysts prepared in Examples 1-7 were subjected to a nitrogen adsorption/desorption test. Resulting nitrogen adsorption/desorption curves are shown in FIG. 2. Specific surface area and average pore size obtained from nitrogen adsorption/desorption test results are listed in Table 1.

Particle strength of the supported catalysts prepared in Examples 1-7 was measured using a particle compressive strength tester. Test results are listed in the Table 1.

Results of sphericity of the supported catalysts prepared in Examples 1-7 are listed in the Table 1.

TABLE 1
Properties of supported catalysts prepared in Examples 1-7

| | Specific | | | |
|---|---|---|---|---|
| | surface | Average | Particle | |
| | area | pore size | strength | |
| Example | m²/g | nm | N/particle | Sphericity |
| Example 1 | 285 | 8.5 | 9.5 | 0.986 |
| Example 2 | 287 | 8.6 | 10.1 | 0.939 |
| Example 3 | 289 | 8.4 | 6.7 | 0.911 |
| Example 4 | 283 | 8.3 | 10.7 | 0.983 |
| Example 5 | 285 | 8.4 | 7.0 | 0.932 |
| Example 6 | 282 | 8.3 | 9.0 | 0.976 |
| Example 7 | 283 | 8.3 | 8.8 | 0.981 |

As can be seen from the results in Table 1, the spherical alumina prepared according to the method provided by the present disclosure exhibits high sphericity; and the supported catalyst prepared by using the spherical alumina as a support has a large specific surface area and high particle strength.

A specific reaction process of using the supported catalysts prepared in Examples 1-7 for a hydrogenation reaction and a dehydrogenation reaction was preformed as follows: the supported catalysts were respectively loaded into a fixed-bed reactor and were reduced and activated by introducing hydrogen under a pressure of 1 MPa and raising a temperature to 350° C. and holding the temperature for 24 hours; and after activation of the supported catalysts was completed, a system pressure was maintained at 0.2 MPa by adjusting a flow rate of the hydrogen, a liquid feed speed was adjusted, and a catalytic dehydrogenation reaction of dibenzyltoluene was conducted at 350° C. (a temperature of the fixed-bed reactor). A gas mass flow meter was connected to a tail end of a gas-phase outlet of the fixed-bed reactor to monitor a instantaneous flow rate of the hydrogen in real time. Based on a feed flow rate, a theoretical hydrogen flow rate after complete dehydrogenation could be calculated, thereby enabling calculation of a hydrogen production rate of the supported catalysts. Results are listed in Table 2.

TABLE 2
Catalytic performance of supported
catalysts prepared in Examples 1-7

| | Hydrogen |
|---|---|
| | production |
| Example | rate (%) |
| Example 1 | 86.07 |
| Example 2 | 87.62 |
| Example 3 | 93.01 |
| Example 4 | 89.17 |
| Example 5 | 94.13 |
| Example 6 | 92.80 |
| Example 7 | 87.12 |

As can be seen from Table 2, the supported catalyst provided by the present disclosure has good catalytic performance and catalytic efficiency for both a hydrogenation reaction and a dehydrogenation reaction in a fixed-bed reactor.

Although the present disclosure is described in detail in conjunction with the foregoing embodiments, they are only a part of, not all of, the embodiments of the present disclosure. Other embodiments could be obtained based on these embodiments without creative efforts, and all of these embodiments shall fall within the scope of the present disclosure.

What is claimed is:

1. A method for preparing a spherical alumina, comprising the following steps:
kneading an alumina powder, an adhesive, and water, and subjecting a resulting mixture to extrusion to obtain a strip-shaped sample; and
subjecting the strip-shaped sample to spheronization to obtain spherical particles, and subjecting the spherical particles to a first calcination to obtain the spherical alumina.

2. The method of claim 1, wherein the adhesive comprises one selected from the group consisting of an inorganic acid, formic acid, acetic acid, and citric acid;
a mass ratio of the alumina powder to the adhesive is in a range of 100: 0.5-6; and
a mass ratio of the alumina powder to the water is in a range of 1:0.5-2.0.

3. The method of claim 1, wherein the extrusion is performed by using a single-screw extruder with auto-feed compression; and
the strip-shaped sample has a diameter of not less 0.6 millimetres (mm).

4. The method of claim 1, wherein the spheronization is performed by placing the strip-shaped sample into a spheronizer and subjecting the strip-shaped sample to spheronization to obtain the spherical particles.

5. The method of claim 1, wherein the first calcination is conducted at a temperature of 500° C. to 600° C. for 3 hours to 6 hours.

6. A spherical alumina prepared by the method of claim 1, wherein the spherical alumina has an average diameter of not less 0.6 mm, a sphericity of 0.911 to 0.986, and a strength of 6.7 N/particle to 10.7 N/particle.

7. A method of using the spherical alumina of claim 6, comprising using the spherical alumina as a catalyst support.

8. A supported catalyst, comprising a support and a catalytically active component loaded on the support, wherein the support is the spherical alumina of claim 6, and the catalytically active component comprises a noble metal; and
a loading amount of the catalytically active component in the supported catalyst is 0.5 wt % to 4 wt %.

9. A method for preparing the supported catalyst of claim 8, comprising the following steps:
impregnating the spherical alumina as the support with a noble metal salt solution by an incipient wetness impregnation process to obtain a spherical alumina loaded with a noble metal salt; and
subjecting the spherical alumina loaded with the noble metal salt to a second calcination to obtain the supported catalyst.

10. A method of using the supported catalyst of claim 8, comprising using the supported catalyst in a hydrogenation reaction or a dehydrogenation reaction in a fixed-bed reactor.

11. The spherical alumina of claim 6, wherein the adhesive comprises one selected from the group consisting of an inorganic acid, formic acid, acetic acid, and citric acid;
a mass ratio of the alumina powder to the adhesive is in a range of 100: 0.5-6; and
a mass ratio of the alumina powder to the water is in a range of 1:0.5-2.0.

12. The spherical alumina of claim 6, wherein the extrusion is performed by using a single-screw extruder with auto-feed compression; and
the strip-shaped sample has a diameter of not less 0.6 mm.

13. The spherical alumina of claim 6, wherein the spheronization is performed by placing the strip-shaped sample into a spheronizer and subjecting the strip-shaped sample to spheronization to obtain the spherical particles.

14. The spherical alumina of claim 6, wherein the first calcination is conducted at a temperature of 500° C. to 600° C. for 3 hours to 6 hours.
